(19) United States
(12) Patent Application Publication
(10) Pub. No.: US 2025/0025120 A1
(43) Pub. Date: Jan. 23, 2025

(54) SYSTEM AND METHOD FOR IN VIVO ESTIMATION OF BRAIN AMYLOID BURDEN USING X RAYS

(71) Applicants: Aldo Badano, Silver Spring, MD (US); Eshan Dahal, Silver Spring, MD (US); Jerome Carson Smith, Crofton, MD (US); Bahaa Ghammraoui, Silver Spring, MD (US)

(72) Inventors: Aldo Badano, Rockville, MD (US); Eshan Dahal, Silver Spring, MD (US); Jerome Carson Smith, Crofton, MD (US); Bahaa Ghammraoui, Silver Spring, MD (US)

(21) Appl. No.: 18/713,085

(22) PCT Filed: Nov. 21, 2022
(86) PCT No.: PCT/US2022/050612

§ 371 Date: May 23, 2024

Related U.S. Application Data
(60) Provisional application No. 63/264,571, filed on Nov. 24, 2021.

(51) Int. Cl. A61B 6/00; A61B 6/50

(52) U.S. Cl. CPC A61B 6/483; A61B 6/501

(57) ABSTRACT

Energy-resolved spectral small angle x-ray scattering (sSAXS) is used to assess in vivo tissues, and in particular, to assess brain amyloid burden. Detected scattering is binned based on a momentum transfer parameter for a plurality of x-ray photon energies. Values with a selected range of momentum transfer parameters are processed to provide the assessment. In examples, the assessment is presented as a displayed image along with a traditional CT image in a sSAXS-CT setup. The CT image can also be used to identify reference paths that tend to lack appreciable amyloid burden.

### FIELD

The disclosure pertains to in vivo measurement and imaging using small angle x-ray scattering.

### BACKGROUND

Amyloid plaque in the brain is associated with a wide range of neurodegenerative diseases such as Alzheimer's and Parkinson's and is defined as aggregates of amyloid fibrils rich in R-sheet structures. Unfortunately, the current approach to detection of amyloid plaque in the in vivo brain are unsatisfactory. For example, positron emission tomography (PET) can be used with amyloid specific radioactive tracers. Estimation of brain amyloid load is limited by variation in the uptake and binding of the tracer. Thus, not only are PET-based approaches complicated by the need for tracers, but the tracers themselves introduce significant uncertainties to any amyloid load measurements. Other approaches such as magnetic resonance imaging (MRI) can require a gadolinium contrast agent and have low specificity. Overall brain amyloid burden has also been estimated in humans by measures of amyloid beta protein in the cerebrospinal fluid and blood plasma. These estimates do not provide information regarding the location in the brain from which the amyloid beta protein originated, thereby limiting their utility in clinical diagnosis or prognosis. Alternative approaches are needed that can provide location information and avoid the use of tracers or contrast agents for in vivo assessment of the human brain.

### SUMMARY

Disclosed herein are methods that comprise irradiating a region of interest (ROI) with a collimated, polychromatic x-ray beam and in response to the irradiating, obtaining energy- and angle-resolved scattering intensities associated with a reference path and a measurement path. The energy- and angle-resolved scattering intensities can be processed to produce scattering cross section as a function of a momentum transfer parameter which can be combined within a predetermined range to determine amyloid burden or otherwise assess a tissue. The methods are suitable for use in vivo and in conjunction with CT scanning.

Representative apparatus comprise an x-ray source operable to produce a collimated, polychromatic x-ray beam and direct the collimated, polychromatic x-ray beam to a specimen. An x-ray detector is situated to receive scattered portions of the collimated, polychromatic x-ray beam from the specimen and produce signals corresponding to energy- and angle-resolved scattering intensities associated with a measurement path and a reference path through the specimen. Logic such as a microprocessor or other device can be configured to process the signals and produce an estimation of a protein aggregate load from a scattering cross-section dependence on a momentum transfer parameter based on the signals corresponding to the energy- and angle-resolved scattering intensities associated with the measurement path and the reference path. The apparatus can be provided in a CT system or otherwise provided.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 illustrates a representative spectral small angle x-ray scattering (sSAXS) system.

FIGS. 1A-1B illustrate representative detector arrangements for use with the disclosed systems and methods.

FIGS. 1B1-1N2 illustrate additional detector arrangements.

FIGS. 1C-1F illustrate signal processing use to estimate the amount of protein aggregates in tissue, using an outcome measure such as amyloid load or amyloid burden based on sSAXS measurements with a pixelated x-ray detector.

FIG. 2A illustrates a representative SAXS system implemented in conjunction with an x-ray computerized tomography (CT) system.

FIG. 2B illustrates combining a CT image with an sSAXS image.

FIG. 3 illustrates another representative SAXS system implemented in conjunction with an x-ray computerized tomography (CT) system.

FIG. 4 illustrates an x-ray source adapted for use in the disclosed systems and methods.

FIG. 5 illustrates a representative method for amyloid load determination using sSAXS.

FIG. 6 illustrates a representative method for combining CT and sSAXS.

FIG. 7 illustrates selection of a reference path and a measurement path in an in vivo human brain.

FIG. 8 illustrates a computing environment for control of sSAXS, CT, and other measurement systems and processing of acquired image and other data.

FIG. 9 illustrates an additional representative system.

FIG. 10 illustrates a representative sample with a sandwiched target (caffeine) in thick objects (PMMA) at centimeter-scale for illustrating the disclosed technology.

FIGS. 11A-11B illustrate relative scattering cross-section as a function of q and area under the peak (AUP), respectively, for a caffeine target sandwiched in 4 cm, 10 cm, and 16 cm of PMMA and a caffeine target without PMMA for reference at an x-ray tube voltage of 80 kVp, a tube current of 1 mA, and an exposure time of 300 s.

FIGS. 12A-12B illustrate relative scattering cross-section as a function of q and area under the peak (AUP), respectively, for a caffeine target sandwiched in 4 cm, 10 cm, and 16 cm of PMMA and without PMMA, at an x-ray tube voltage of 140 kVp, a tube current of 1 mA, and an exposure time of 300 s.

FIGS. 13A-13B illustrate relative scattering cross-section as a function of q and area under the peak (AUP), respectively, for a caffeine target sandwiched in 10 cm of PMMA at an x-ray tube voltage of 80 kVp, and tube current-time product values of 300 mAs, 900 mAs, and 1500 mAs with a caffeine target without PMMA as a reference at 300 mAs

FIGS. 14A-14B illustrate relative scattering cross-section as a function of q and area under the peak (AUP), respectively, for a caffeine target sandwiched in 10 cm of PMMA and a caffeine target without PMMA, at an x-ray tube voltage of 140 kVp, and tube current-time product values of 60 mAs and 300 mAs.

### DETAILED DESCRIPTION

### Introduction and Terminology

In the following, SAXS systems and methods are described with reference to determination of amyloid load in the human brain in vivo. However, the disclosed approaches can be applied to other subjects, other tissues with various protein deposits, or in the evaluation of other tissue abnormalities or features such as tau and phosphorylated tau protein. As used herein, subject or tissue characteristic is used to refer to any specimen features of interest. Amyloid load determinations are described as based on an area under a scattering cross-section curve in a selected range and is referred as an area under the peak (AUP) determination. However, generally amyloid load determination can be based on a combination of one or more scattering cross section values in a selected range, such as an average of some or all values in the selected range. Visual display of scattering cross-section values is not necessary.

Scattering cross-section refers to scattering intensity as a function of a momentum transfer parameter, defined herein as q=4πE sin θ/hc, wherein E is x-ray energy, θ is a scattering angle measured from an x-ray beam axis or propagation, and hc is a product of Planck's constant and the vacuum speed of light. As used herein, a momentum transfer parameter q is a variable that is proportional to a product of E and sin θ (or E and θ since a small angle approximation is generally adequate). The momentum transfer parameters that are scaled or defined differently than q will have different values and units but correspond to the q values. In terms of a momentum transfer parameter defined as q=4πE sin θ/hc, a tissue characteristic can be evaluated based on ranges of q values. For amyloid load ranges of q (in nm⁻¹) can be 3-8, 3.5-8.5, 4-8, 7.3-9.7, 7-10, 8-10, 10-20, 0.5-30 or other q-ranges.

In the disclosed examples, scattered x-rays are detected at angles of less than 20, 15, 10, 5, or 2 degrees from an unscattered beam axis, and typically in an angular cone having an angular radius of 20, 15, 10, 5, or 2 degrees. In some examples, scattered radiation is measured in only a portion of an angular cone such as one-half, one-quarter, or less of an angular cone. Irradiation can be with x-ray beams having photon energies in ranges corresponding to ranges used in diagnostic imaging as well as other ranges such 50-120 keV or 30-80 keV to maximize SAXS signal. As used herein, a collimated x-ray beam has an angular radius of less than 10, 5, 2, 1, 0.5, or 0.1 degrees. In convenient examples, a beam angular radius is small compared to a maximum scattering angle to be used, such as a beam angular radius that is less than ½, ⅕, 1/10, 150, 1/100, 1/200, or 1/500 times a maximum scattering angle. Beam diameters are typically less than 10 mm, 5 mm, 4 mm, 3 mm, 2 mm, or 1 mm and in some examples diameters between 3 mm and 5 mm are selected. The numbers of beams, and their shape and size can be changed accordingly depending on the resolution and other measurement requirements. Measured scattered x-ray intensity as a function of scattering angle θ and photon energy E (referred to herein as Iₘₑₐₛ(θ, E)) can be obtained with one or more x-ray detectors although detector arrays generally provide simpler implementations. In some examples, a so-called hyperspectral, photon-counting pixelated area detector is used that provides scattered x-ray intensity as a function of photon energy for a plurality of locations as defined by pixel size. In typical example, the sensor is based on a CdTe crystal.

A region of interest (ROI) can be irradiated at different angles to obtain corresponding scattered intensities Iₘₑₐₛ(θ, E) for corresponding paths and then tomographically processed to obtain an image of the ROI associated with scattering or with a tissue characteristic. The scattering or tissued characteristic image can be displayed along with a CT image which can be at a higher resolution if desired. In addition, the CT image can be used to identify portions of the ROI that can be used to obtain one or more reference scattering intensities I_{ref}(θ, E) to compensate measured scattering intensities, if needed. In some examples, transmittance of an x-ray beam through a specimen as a function of photon energy is sufficient to provide compensation. Signals and scattering intensities as used herein can refer to digital or other numerical representations that can be communicated over a computer network or to time-varying voltages or currents that can be, for example, directed to an analog-to-digital convertor (ADC) to produce an associated digital representation. ADCs can be situated at detectors or at control circuitry or data processors as convenient. As used herein, image can refer to a visual presentation on a display device for a use by a technician or a digital representation that can be stored and processed in DICOM or other formats such as JPEG, TIFF, PDF or in video formats such as MPEG. Quantitative values can also be displayed to aid in the interpretation of the case.

In some disclosed examples, the estimation of the amyloid burden/load is done through combining information obtained through different x-ray beams at different angles targeting a region of interest via sSAXS. Because of limited sampling, this 3D image reconstruction will generally be low resolution compared to an anatomical CT image. In the examples, particular arrangements of detectors are used, but other configuration of detectors can be used that provide scattering intensity as a function of angle and x-ray photon energy.

In the examples below, various processing and control systems can be used to control data acquisition and evaluate the acquired data. For convenience, the term “logic” is used to refer to processors such as microprocessors, application specific integrated circuits (ASICs), FPGAs, and other general purpose or dedicated processing hardware and the associated storage and executable instructions. For clarity of description, such logic is also referred as a controller or control system.

As used in this application and in the claims, the singular forms “a,” “an,” and “the” include the plural forms unless the context clearly dictates otherwise. Additionally, the term “includes” means “comprises.” Further, the term “coupled” does not exclude the presence of intermediate elements between the coupled items.

The systems, apparatus, and methods described herein should not be construed as limiting in any way. Instead, the present disclosure is directed toward all novel and non-obvious features and aspects of the various disclosed embodiments, alone and in various combinations and sub-combinations with one another. The disclosed systems, methods, and apparatus are not limited to any specific aspect or feature or combinations thereof, nor do the disclosed systems, methods, and apparatus require that any one or more specific advantages be present or problems be solved. Any theories of operation are to facilitate explanation, but the disclosed systems, methods, and apparatus are not limited to such theories of operation.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed systems, methods, and apparatus can be used in conjunction with other systems, methods, and apparatus. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art.

In some examples, values, procedures, or apparatus are referred to as “lowest”, “best”, “minimum,” or the like. It will be appreciated that such descriptions are intended to indicate that a selection among many used functional alternatives can be made, and such selections need not be better, smaller, or otherwise preferable to other selections.

Examples are described with reference to directions indicated as “above,” “below,” “upper,” “lower,” and the like. These terms are used for convenient description, but do not imply any particular spatial orientation.

### Example 1

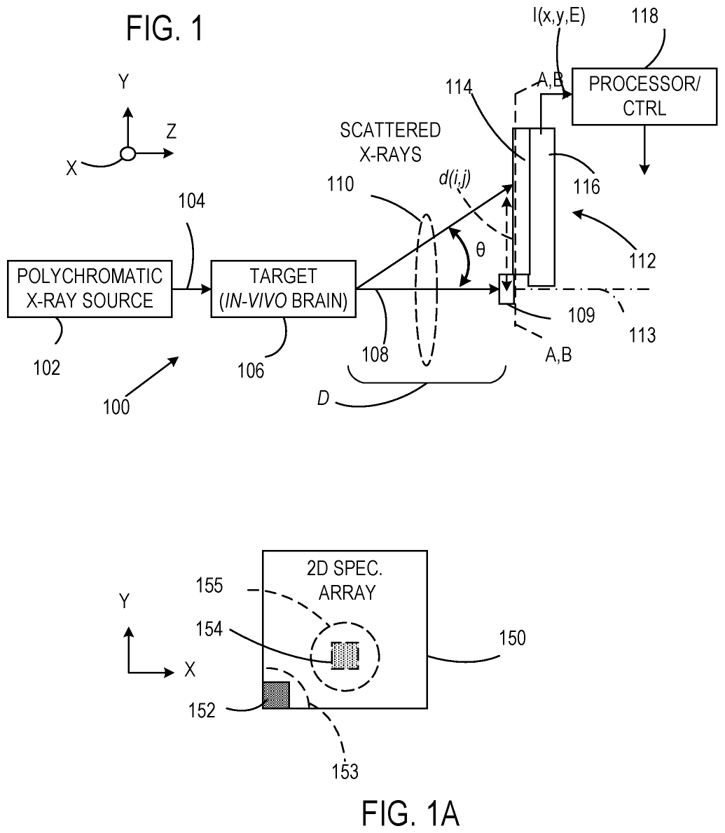

With reference to FIG. 1, an sSAXS system 100 includes a polychromatic x-ray source 102 that provides a collimated x-ray beam 104 that is directed to a target 106. In one example, the target 106 is an in vivo human brain. The collimated x-ray beam 104 is scattered at the target 106 to produce a scattered beam 110 portions of which are directed to an x-ray detector 112 that includes a detector sensor 114 such as one made from CdTe and processing electronics 116 defined in an application-specific integrated circuit (ASIC) mounted on an aluminum block. Unscattered portions of the x-ray beam 104 are blocked or partially transmitted by a beam stop 109. In this example, the x-ray detector 112 is a hyperspectral pixeled detector having an 80 by 80 array of detector pixels defined on a 250 μm pitch. Each pixel is operable to produce a charge proportional to incident photon energy so that each pixel can be read-out to obtain a spectral distribution of the scattered beam 110 at an associated scattering angle that can be digitally communicated via a network such as a local area or other network. Thus, the x-ray detector 112 thus provides scattering intensities I_{ref}(θ, E) for a plurality of energies and angles (i.e., 6400 different angular locations) as a number of counts as a function of energy for each detector pixel.

In FIG. 1, the x-ray detector 112 is situated to receive scattered x-rays in an angular range on one side of an x-ray beam axis 113 but can be situated to receive scattered x-rays in other angular ranges. FIGS. 1A-1B are sectional views illustrating representative detector arrangements. Referring to FIG. 1A, a two-dimensional pixelated spectral detector 150 having detector pixels that are distributed along an X-direction and a Y-direction is shown along with representative unscattered beam stop locations 152, 154. As shown, arc 153 shows locations associated with a selected scattering angle with the detector 150 aligned with the beam stop location 152 and circle 155 shows locations associated with a selected scattering angle with the detector 150 aligned with the beam stop location 154. In some examples, it can be convenient to position a detector so that an unscattered beam stop location corresponds to a center of the detector 150. Referring to FIG. 1B, a representative x-ray detector 160 includes two dimensional pixelated detectors 162-164 and an associated unscattered beam stop location is shown at 168. Circle 169 illustrates locations associated with a selected scattering angle. One, two, or more such two-dimensional detectors can be used and they can be situated as illustrated or in other configurations as convenient. Array detectors are not necessary and one or more discrete detectors can be used if desired and one-dimensional arrays can be used as well. FIGS. 1B1-1B2 illustrate x-ray detectors 161, 159, respectively, that are similar to that of FIG. 1B but with two of the detector arrays omitted. Other arrangements of one or more detectors or detector arrays can be used, and only selected examples are shown in the drawings.

Processing of the acquired scattering intensities Iₘₑₐₛ(θ, E) is illustrated in FIGS. 1C-1E. FIG. 1C illustrates a number of detected counts N as a function of location (i,j) on the pixelated sensor 112 for four different x-ray energies E between 30 and 45 keV, i.e., N(i,j,E), as frames 171-174, wherein i, j are positive integers. For an 80 pixel by 80 pixel detector, i, j each range from 0 to 79. Pixel locations i,j can be used to establish a scattering angle based on a distance D from the scattering and a distance d(i,j) from the beam axis to the detector pixel indicated by i,j. N(i,j,E) thus corresponds to Iₘₑₐₛ(θ, E). Beam transmission from an area in which the feature of interest is lacking can be used to provide a transmittance correction Nₜ(E), wherein Nₜ represents a number of detector counts associated with the unscattered beam. In other examples, numbers of counts as a function of position in a reference sample can be used to provide a transmittance correction, i.e., Nₜ(i,j,E) which corresponds to I_{ref}(θ, E). A corrected or compensation number of counts N_{comp}(i,j,E) is produced as a ratio Nₜ(i,j, E)/Nₜ(E).

The compensated counts are then summed into bins based on associated values of q, i.e., bins that have a product of E and θ with a specified bin width. For an array detector, detector pixel locations can be used to determine θ. In an example in FIG. 1AA, an unscattered beam is directed toward a pixel 195 denoted as (0,0) and a distance of a pixel dᵢⱼ from the beam axis is sqrt[(iΔx)²+(jΔy)²], wherein Δx and Δy are pixel spacings along an x-axis and a y-axis, respectively. Scattering angles for each detector pixel are given by θ(i,j)=tan⁻¹(dᵢⱼ/D), wherein D is a distance from the scattering or target-to-detector distance as shown in FIG. 1. Using such geometrically determined values of scattering angle, values of N_{comp}(i,j,E) can be summed in bins of a specified q width such as Δq=1.2 nm⁻¹ or other values and the values combined for all values of E in the energy range of interest. FIG. 1D shows curves 181-184 showing binning of data from frames 171-174 to produce scattering cross-section S(q) for associated x-ray energy values which are then combined in curve 190 shown in FIG. 1E. An area under the peak (AUP) 192 is computed for the q-range [3.6 nm⁻¹, 8.4 nm⁻¹] as an indicator for use in amyloid load estimation.

### Example 2

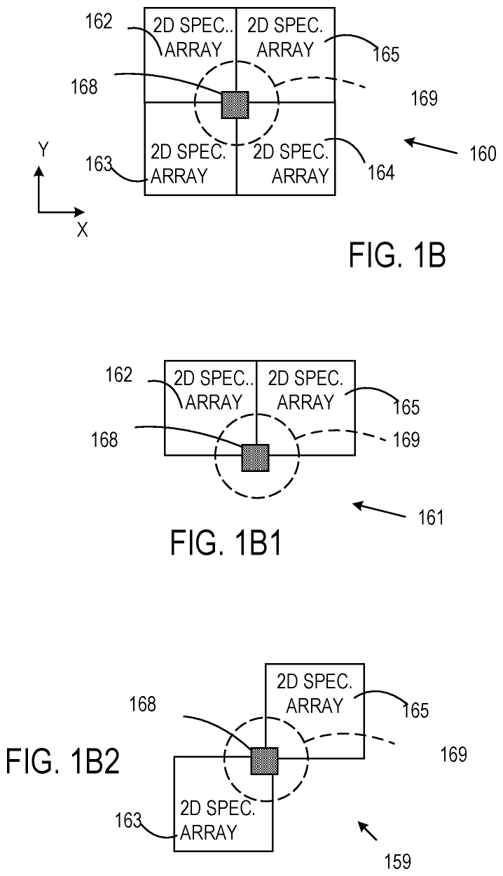

Referring to FIG. 2A, a CT scanning system 200 includes a gantry 202 that is operable to rotate an x-ray source 204 and one or more x-ray detectors 206 about a scanning region 208 as shown at 210. A subject can be situated on a Z-axis stage 212 to be moved along a Z-axis of a coordinate system 214. In FIG. 2, the Z-axis is perpendicular to the plane of the drawing.

A CT controller 220 is coupled to the gantry 202, the x-ray source 204, the one or more x-ray detectors 206, and the Z-axis stage 212 to irradiate a subject situated in the scanning region 208 with x-rays along multiple directions with the subject at multiple Z-axis locations and record corresponding x-ray signals. In the example of FIG. 2, the x-ray source 204 produces a fan beam. X-rays detected by the one or more x-ray detectors 206 produce signals that are directed to the CT controller 220 for storage or communication over a network. These CT signals from multiple detectors at multiple rotation angles of the gantry 202 can be tomographically processed to form an image of a subject situated in the scanning zone 208. The image can be stored and presented on a display device 221.

The CT scanning system 200 can also include a polychromatic, collimated x-ray source 220 that is situated to direct a polychromatic, collimated x-ray beam 222 to the scanning zone 208 so that scattered beam portions 224 from a subject are directed to a two-dimensional pixelated spectral detector 226 while an unscattered portion 228 is directed to a beam stop 230. The two-dimensional pixelated spectral detector 226 provides scattering intensities Iₘₑₐₛ(θ, E) in response to the x-ray irradiation and these scattering intensities are also coupled to the CT controller 220. These scattering intensities can be tomographically processed as discussed above to provide an image associated with loading of the subject by a feature of interest, such as amyloid load in an in vivo human brain. This image can also be displayed on the display device 221 along with CT image. Images can correspond to the scattering intensities or to scattering cross-sections S(q) as discussed above.

In many cases, it is desirable to compensate the scattering intensities for scattering and attenuation in the subject that are unrelated to the feature of interest. The CT image can be used to select subject areas that are known or likely to have low or no load associated with the features of interest. Upon acquisition of the CT image, a series of rotations of the collimated x-ray source 220 and the two-dimensional pixelated spectral detector 226 can be used to obtain scattering intensities Iₘₑₐₛ(θ, E) and I_{ref}(θ, E) along measurement paths and one or more reference paths, respectively. In some cases, a single or few reference paths suffice for compensation of Iₘₑₐₛ(θ, E). As discussed above, it is generally sufficient to determine transmittance as a function of x-ray energy for a reference path to compensate measured scattering intensity. The controller 220 receives Iₘₑₐₛ(θ, E) and I_{ref}(θ, E) (or merely I_{ref}(E)) and processes to determine scattering cross-section S as a function of a scattering parameter. i.e., S(q). Values of S(q) in a selected range can be combined such as by integration and specimen loading or other specimen characteristic assessed based on the combination. A particular energy range can be selected in view of higher tissue absorption at lower energies and increased x-ray output and target-to-detector distances at higher energies. In the example of FIG. 2, x-ray energies are typically between 30-80 keV for amyloid burden estimation.

FIG. 2B illustrates a representative image shown a CT image 250 with an associated amyloid burden image 252. Typically, a resolution of the image 252 is less than that of the CT image 250.

### Example 3

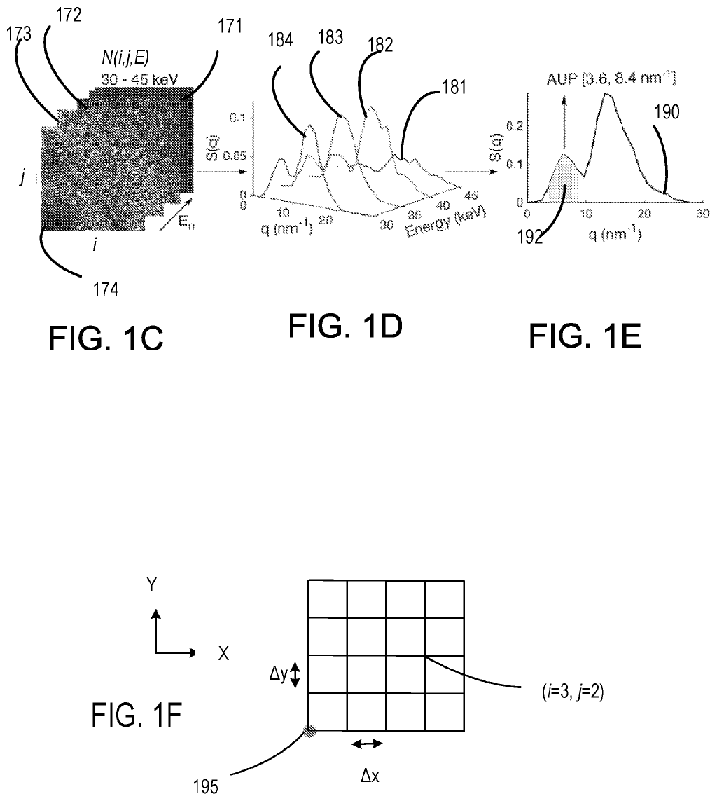

Referring to FIG. 3. A combined CT/sSAXS system 300 includes a gantry 302 that is operable to rotate an x-ray source 304 and an oppositely situated x-ray detector array 306 about a scanning region 308. A z-axis stage 310 is situated to translate a subject with respect to the gantry 302. A collimator 312 can be switchably coupled to the x-ray source 304 so that beams suitable for CT scanning and sSAXS can be produced with a single x-ray source. A controller 314 is coupled to the gantry 302, the x-ray source 304, the x-ray detector array 306, the z-axis stage 310 to select angles of rotation, activate irradiation, establish x-ray beam configuration, receive detector data, and otherwise provide control and data computation. The combined CT/sSAXS system 300 is similar to that of FIG. 2, but in the in the FIG. 3 example, a single x-ray source and a single detector array are used for both CT scanning and sSAXS measurements. In other examples, different sources and/or different detectors can be used. As shown in FIG. 3, a beam from the x-ray source 304 can be provided as a collimated beam or as a fan beam or other beam shape to provide CT imaging. The x-ray source 304 can also be controlled to provide different x-ray photon energies for CT imaging and sSAXS measurements.

### Example 4

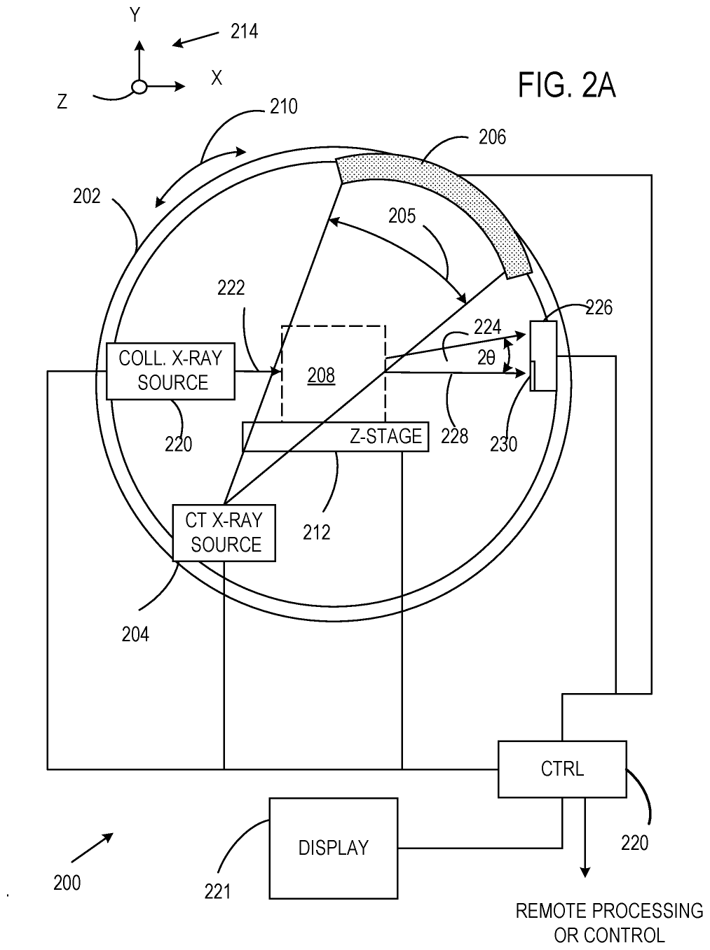

Referring to FIG. 4, an x-ray source 402 is situated to direct a diverging x-ray beam 404 to a one or more collimating aperture plates 406 to produce a collimated x-ray beam 412. The one or more aperture plates 406 are insertable or removable from the beam path of the x-ray source 402 with an actuator 408 that can translate the aperture plates 406 along a direction 410. In this way, an uncollimated CT x-ray beam can be made suitable for sSAXS. The x-ray source 302 can also be driven at different voltages or otherwise configured to produced different x-ray photon energy distributions in the CT and sSAXS beams.

### Example 5

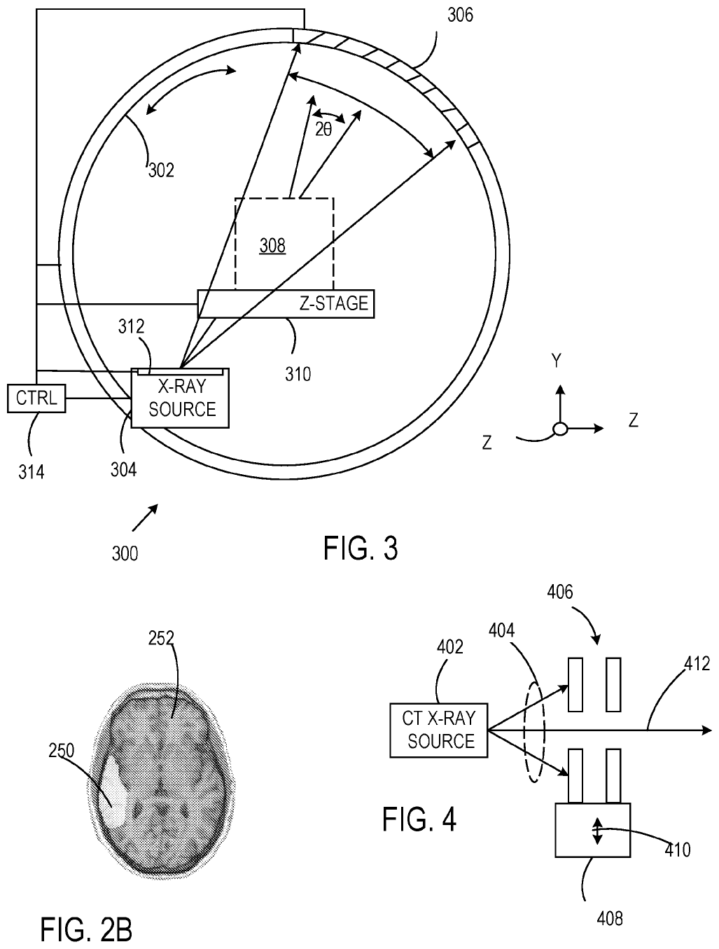

Referring to FIG. 5, a representative method 500 includes selecting sSAXS x-ray energy range, scattering angles, and momentum transfer parameter ranges at 502. At 504, one or more reference x-ray beam paths in a subject under investigation are selected and corresponding spectral transmittances are determined. At 506, spectral scattering intensities of along one or more measurement paths are measured in the selected scattering angle and x-ray energy ranges. At 508, the measured spectral scattering intensities are compensated based on measurements associated with the reference paths. At 510, the compensated spectral scattering intensities are processed to establish scattering cross-section as a function of a momentum transfer parameter for the x-ray energies used. At 512, the scattering cross-sections are combined (at least within the selected momentum transfer parameter range) and at 514 a subject characteristic such amyloid load is estimated based on the combination. If additional subject locations are determined to be of interest at 516, processing returns to 504 although in some cases, additional reference paths are not needed, and processing can return to 506.

### Example 6

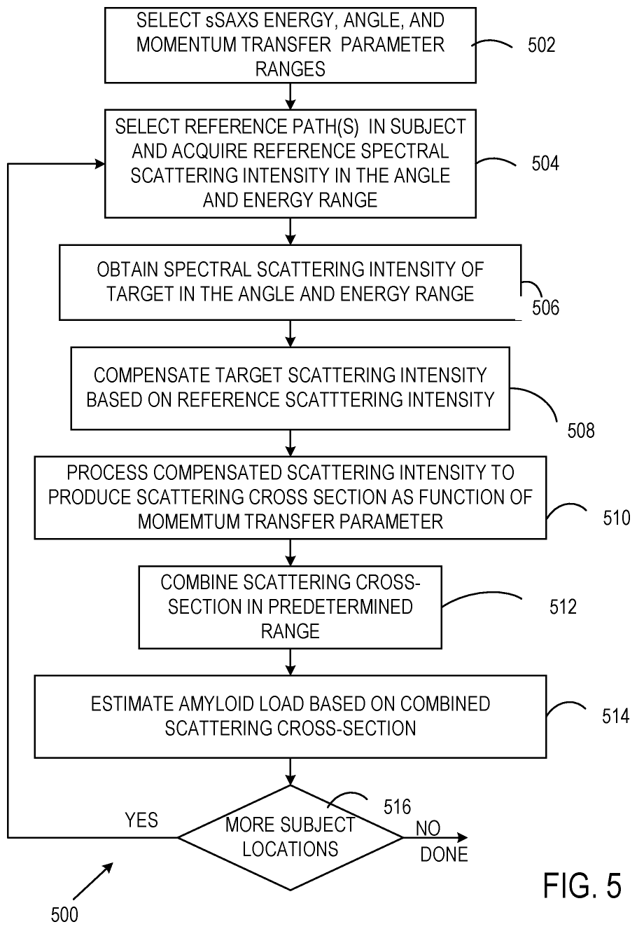

Referring to FIG. 6, a representative method 600 includes obtaining a CT image of a region of interest (ROI) at 602. At 604, one or more reference x-ray beam paths and measurement beam paths in the ROI are selected based on the CT image. At 606, the x-ray beam source and detector are rotated to irradiate the ROI along the reference and measurement paths, and respective spectral scattering intensities are measured. In some cases, only the spectral transmittance along a single reference path is sufficient for compensation or compensation is unnecessary. At 608, the measured spectral scattering intensities are compensated based on measurements associated with the reference paths (or path) and at 610, the compensated spectral scattering intensities are processed to establish scattering cross-section as a function of a momentum transfer parameter for the x-ray energies used. At 612, the scattering cross-sections are combined (at least within the selected momentum transfer parameter range) to estimate a target load or amount such amyloid load. At 614, the target loads along the measurement paths (such as amyloid load) are tomographically processed to produce a corresponding target load image. At 616, the CT image and the target load image can be combined and presented on a display device.

### Example 7

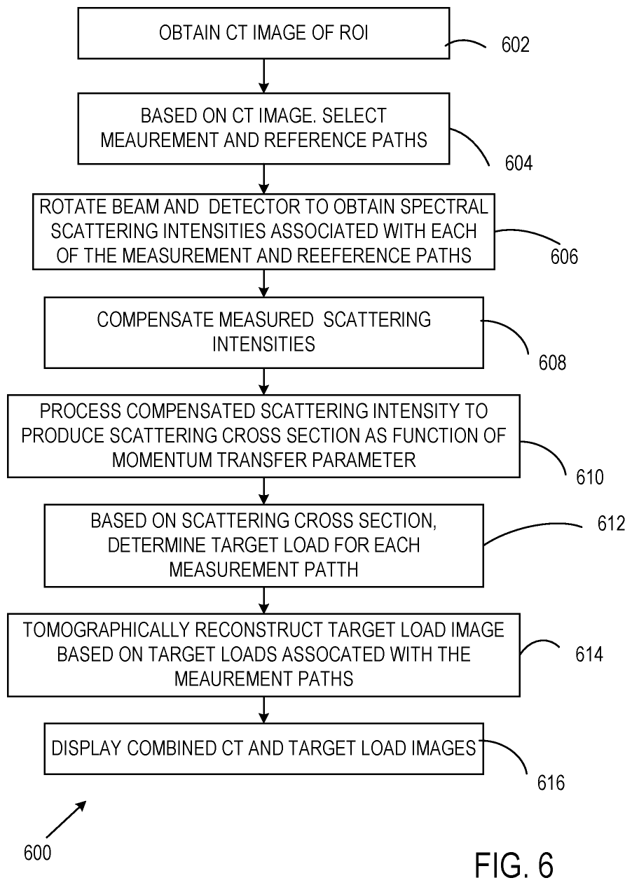

FIG. 7 illustrates finding a reference path using a CT image 710 of an in vivo brain. An x-ray source 702 directs an x-ray beam along a reference path 704 and a measurement path 706. The reference path 704 is spaced away from an amyloid loaded region 708 but is within an in vivo brain 710. As shown above, visualization of the information on amyloid load can be displayed as an overlay on top of a grayscale anatomical CT image.

### Example 8

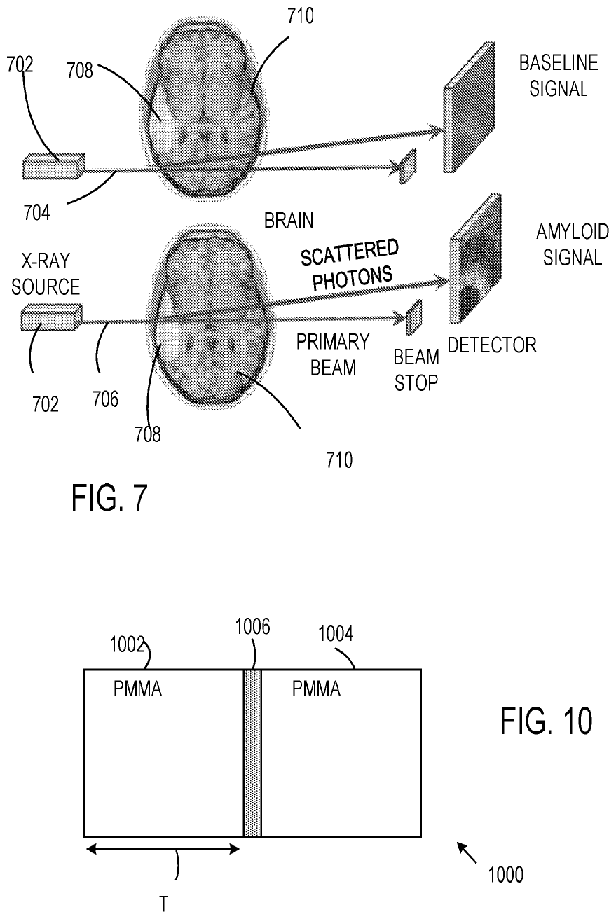

FIG. 8 and the following discussion are intended to provide a brief, general description of an exemplary computing environment in which the disclosed technology may be implemented. Other logic can be used as noted above. Although not required, the disclosed technology is described in the general context of computer executable instructions, such as program modules, being executed by a personal computer (PC). Generally, program modules include routines, programs, objects, components, data structures, etc., that perform particular tasks or implement particular abstract data types. Moreover, the disclosed technology may be implemented with other computer system configurations, including handheld devices, multiprocessor systems, microprocessor-based or programmable consumer electronics, network PCs, minicomputers, mainframe computers, and the like. The disclosed technology may also be practiced in distributed computing environments where tasks are performed by remote processing devices that are linked through a communications network. In a distributed computing environment, program modules may be located in both local and remote memory storage devices.

With reference to FIG. 8, an exemplary system for implementing the disclosed technology includes a general-purpose computing device in the form of an exemplary conventional PC 800, including one or more processing units 802, a system memory 804, and a system bus 806 that couples various system components including the system memory 804 to the one or more processing units 802. The system bus 806 may be any of several types of bus structures including a memory bus or memory controller, a peripheral bus, and a local bus using any of a variety of bus architectures. The exemplary system memory 804 includes read only memory (ROM) 808 and random-access memory (RAM) 810. A basic input/output system (BIOS) 812, containing the basic routines that help with the transfer of information between elements within the PC 800, is stored in ROM 808. The memory 804 also contains portions 871-873 that include processor-executable instructions for cross-section processing and q-binning, path selection, and data acquisition, respectively, as well as processor-executable instructions for tomographic image reconstruction.

The exemplary PC 800 further includes one or more storage devices 830 such as a hard disk drive for reading from and writing to a hard disk, a magnetic disk drive for reading from or writing to a removable magnetic disk, and an optical disk drive for reading from or writing to a removable optical disk (such as a CD-ROM or other optical media). Such storage devices can be connected to the system bus 806 by a hard disk drive interface, a magnetic disk drive interface, and an optical drive interface, respectively. The drives and their associated computer readable media provide nonvolatile storage of computer-readable instructions, data structures, program modules, and other data for the PC 800. Other types of computer-readable media which can store data that is accessible by a PC, such as magnetic cassettes, flash memory cards, digital video disks, CDs, DVDs, RAMs, ROMs, and the like, may also be used in the exemplary operating environment.

A number of program modules may be stored in the storage devices 830 including an operating system, one or more application programs, other program modules, and program data. A user may enter commands and information into the PC 800 through one or more input devices 840 such as a keyboard and a pointing device such as a mouse. Other input devices may include a digital camera, microphone, joystick, game pad, satellite dish, scanner, or the like. These and other input devices are often connected to the one or more processing units 802 through a serial port interface that is coupled to the system bus 806 but may be connected by other interfaces such as a parallel port, game port, or universal serial bus (USB). A monitor 846 or other type of display device is also connected to the system bus 806 via an interface, such as a video adapter. Other peripheral output devices, such as speakers and printers (not shown), may be included.

The PC 800 may operate in a networked environment using logical connections to one or more remote computers, such as a remote computer 860. In some examples, one or more network or communication connections 850 are included. The remote computer 860 may be another PC, a server, a router, a network PC, or a peer device or other common network node, and typically includes many or all of the elements described above relative to the PC 800, although only a memory storage device 862 has been illustrated in FIG. 8. The personal computer 800 and/or the remote computer 860 can be connected to a logical a local area network (LAN) and a wide area network (WAN). Such networking environments are commonplace in offices, enterprise-wide computer networks, intranets, and the Internet.

When used in a LAN networking environment, the PC 800 is connected to the LAN through a network interface. When used in a WAN networking environment, the PC 800 typically includes a modem or other means for establishing communications over the WAN, such as the Internet. In a networked environment, program modules depicted relative to the personal computer 800, or portions thereof, may be stored in the remote memory storage device or other locations on the LAN or WAN. The network connections shown are exemplary, and other means of establishing a communications link between the computers may be used.

### Example 9

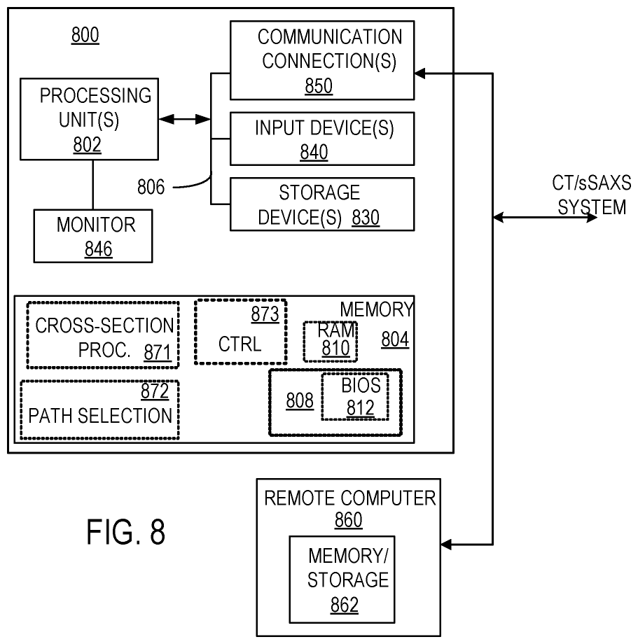

FIG. 9 illustrates are representative apparatus 900 that includes an x-ray source 902 that is coupled to drive electronics 904 that can provide a selected tube current (mA) and tube voltage (kVp). An emitted beam is directed through apertures of 2.5 mm and 2 mm diameter in aperture plates 906, 908, respectively, to a sample 910. Scattered x-rays in a scattered beam 912 are incident to a detector 916 situated a distance D from the sample 910. A maximum scattering angle θₘₐₓ at which scattered x-rays are detected in a function of a detector dimension d and the sample distance D. A beam block 925 is situated to partially block unscattered x-rays. In this example, the sample distance is 290 mm, the detector 916 is a 2D spectroscopic photon-counting x-ray detector that is an 80 by 80 pixel array with 250 μm pixels based on a 1 mm thickness of CdTe. In the examples below, scattering data is acquired for between 60 s and 300 s with 1 keV energy bins. The x-ray source 902 includes a tungsten target and is operated at 80 kVp or 140 kVp with tube current-time product ranging from 60 mAs to 1500 mAs.

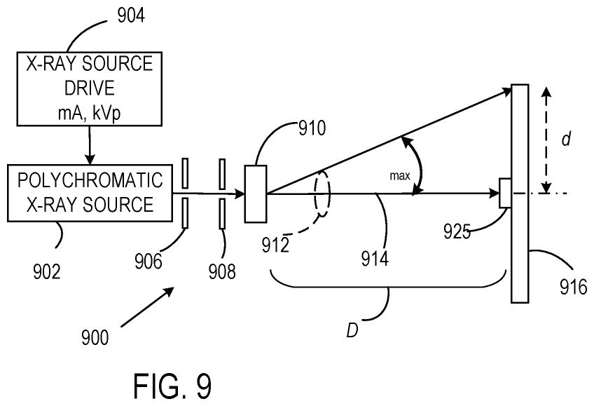

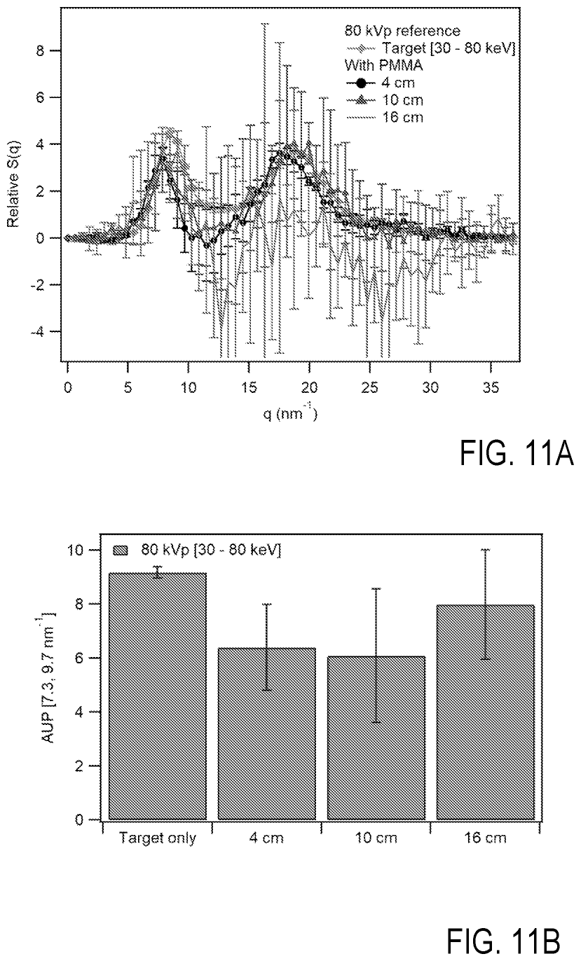

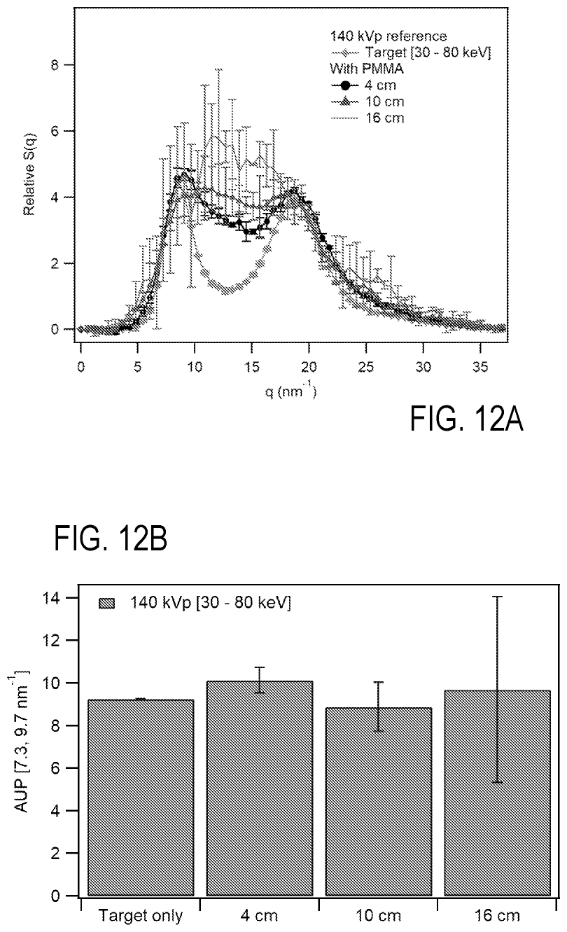

Representative measurements with the apparatus of FIG. 9 can be made using a caffeine target 1006 sandwiched between poly methyl(methacrylate) (PMMA) layers 1002, 1004 as shown in FIG. 10. In the examples discussed below, the layers 1002, 1004 are of equal thicknesses of 2 cm, 5 cm, and 8 cm with the caffeine target located at the middle of the PMMA layers. Unless otherwise noted, scattering cross-section is obtained with an x-ray tube voltage of 80 kVp or 140 kVp and in an x-ray energy range of 30 to 80 keV; AUP is determined using a q range of 7.3 nm⁻¹ to 9.3 nm⁻¹. Other ranges can be used and configured to target specific scattering peaks. In the examples, a scattering peak near 8 nm⁻¹ is used and ranges of 5-10, 6-9, 7-9, 8-9, 3-8, or 4-7 nm⁻¹ or others can be used. As shown in FIG. 11A, thicker samples are associated with higher noise levels but AUP for all thicknesses clearly show measurements that correspond to AUP for the caffeine sample alone (“Target”). FIGS. 12A-12B show results for an x-ray source voltage of 140 kVp. As with FIGS. 11A-11B, AUP corresponds to AUP for the target alone, even for the thickest sample of 16 cm. Noise increases, and a trough in the cross-section curve for the target with PMMA increases with thickness, but AUP determination is successful despite spectral degradation. Higher tube voltage may also result in increased Compton scattering which is not associated with sample evaluation and may not improve AUP measurement.

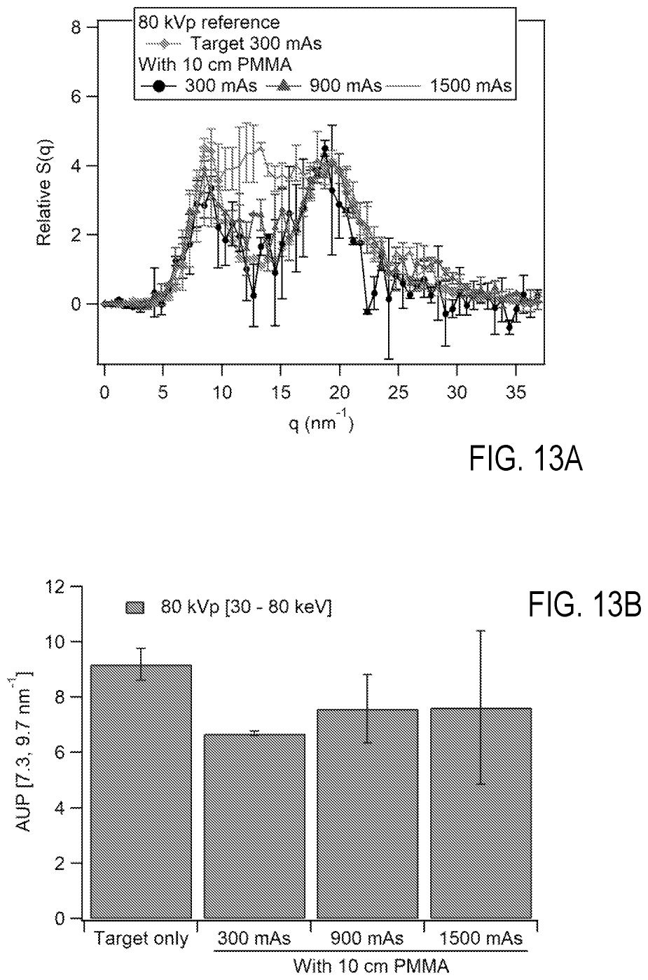

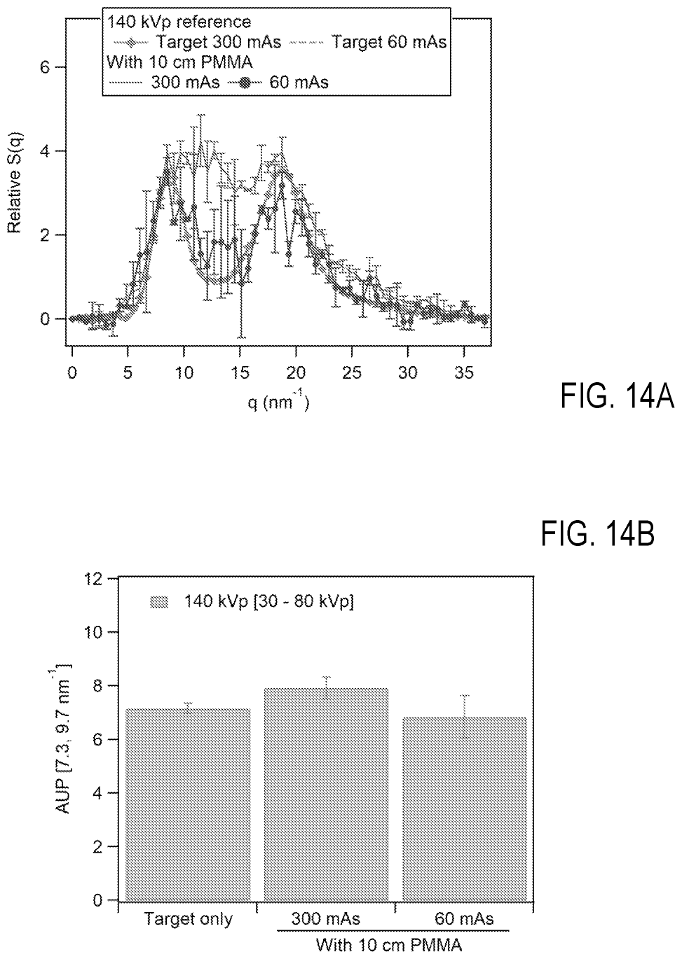

FIGS. 13A-13B illustrate results for a caffeine target situated between two 5 cm thick PMMA layers at an 80 kVp tube voltage and tube current-time product values of 300 mAs, 900 mAs, and 1500 mAs. All mAs permit sample assessment as shown in FIG. 13B, but at 1500 mAs, scattering cross-section has a reduction in the depth of a valley between about 9 nm⁻¹ and 19 nm⁻¹. AUP is determined for an interval that generally avoids this valley. At high mAs, detector spectral response can be degraded with high photon counts. Total scattering is larger but detector response may result in this additional scattering being ineffective to improve measurement accuracy. FIGS. 14A-14B illustrate results for a caffeine sample situated between two 5 cm thick PMMA layers at a 140 kVp tube voltage and tube current-time product values of 60 mAs and 300 mAs. Both mAs permit sample assessment as shown in FIG. 14B, but at 300 mAs, scattering cross-section has a reduction in the depth of a valley between about 9 nm⁻¹ and 19 nm⁻¹, suggesting possible degradation in spectroscopic performance of the detector with high photon flux This spectral degradation in q can be avoided by using low mAs, as demonstrated in FIGS. 14A-14B. While the results of FIGS. 11A-14B can have significant measurement uncertainty as indicated by error bars on the figures, satisfactory measurements can be obtained in all cases by using an improved spectroscopic detector that can handle high flux. While higher energy x-rays can be used beyond 120 keV, the smaller scattering angles can make measurement more difficult in practice.

### Example Embodiments

Embodiment 1 is a method, including: irradiating a region of interest (ROI) with a collimated, polychromatic x-ray beam; in response to the irradiating, obtaining energy- and angle-resolved scattering intensities associated with a reference path and a measurement path; processing the energy- and angle-resolved scattering intensities to produce scattering cross section as a function of a momentum transfer parameter; and combining scattering cross-section within a predetermined range to determine amyloid burden.

Embodiment 2 includes the subject matter of Embodiment 1, and further specifies that the predetermined range of momentum transfer parameter corresponds to momentum transfer parameter values between 3/nm and 9/nm, wherein q=4πE sin θ/hc, wherein E is x-ray energy, θ is a scattering angle, and hc is product of Planck's constant h and a vacuum speed of light c.

Embodiment 3 includes the subject matter of any of Embodiments 1-2, and further specifies that the momentum transfer parameter is q.

Embodiment 4 includes the subject matter of any of Embodiments 1-3, and further includes selecting at least one of the reference path and the measurement path based on a CT image of the ROI.

Embodiment 5 includes the subject matter of any of Embodiments 1-4, and further includes selecting the reference path to avoid portions of the ROI associated with an amyloid load.

Embodiment 6 includes the subject matter of any of Embodiments 1-5, and further includes irradiating the region of interest (ROI) with the collimated, polychromatic x-ray beam along a plurality of measurement paths and for each measurement path: processing associated energy- and angle-resolved scattering intensities to produce scattering cross section as a function of the momentum transfer parameter; and combining each of the scattering cross-sections within the predetermined range to determine amyloid burden along each of the measurement paths.

Embodiment 7 includes the subject matter of any of Embodiments 1-6, and further includes irradiating the region of interest (ROI) with the collimated, polychromatic x-ray beam along reference paths associated with respective measurement paths for each measurement path, wherein the associated energy- and angle-resolved scattering intensities for the measurement paths and the respective reference paths are processed to produce scattering cross sections as a function of the momentum transfer parameter along each of the measurement paths.

Embodiment 8 includes the subject matter of any of Embodiments 1-7, and further specifies that the ROI includes in vivo human brain and the amyloid burden is associated with the in vivo human brain.

Embodiment 9 includes the subject matter of any of Embodiments 1-8, and further specifies that the irradiating the region of interest (ROI) with the collimated, polychromatic x-ray beam along a plurality of measurement paths comprises selecting a rotation of the collimated, polychromatic x-ray source and an oppositely situated x-ray detector about the ROI for each measurement path and obtaining energy- and angle-resolved scattering intensities associated with a reference path and a measurement path.

Embodiment 10 includes the subject matter of any of Embodiments 1-9, and further specifies that the energy- and angle-resolved scattering intensities associated with a reference path and a measurement path are obtained with an x-ray detector array.

Embodiment 11 includes the subject matter of any of Embodiments 1-10, and further specifies that the x-ray detector array comprises a plurality of individual x-ray detectors.

Embodiment 12 includes the subject matter of any of Embodiments 1-11, and further specifies that the CT image is obtained with an uncollimated x-ray beam from an x-ray source, and further includes situating at least one aperture with respect to the x-ray source to produce the collimated, polychromatic x-ray beam.

Embodiment 13 includes the subject matter of any of Embodiments 1-12, and further specifies that polychromatic x-ray beam has x-ray photon energies between 30 and 80 keV.

Embodiment 14 is an apparatus, including: an x-ray source operable to produce a collimated, polychromatic x-ray beam and direct the collimated, polychromatic x-ray beam to a specimen; an x-ray detector situated to receive scattered portions of the collimated, polychromatic x-ray beam from the specimen and produce signals corresponding to energy- and angle-resolved scattering intensities associated with a measurement path and a reference path through the specimen; and logic configured to process the signals and produce an indication of a tissue load from a scattering cross-section dependence on a momentum transfer parameter, wherein the scattering cross section is based on the signals corresponding to the energy- and angle-resolved scattering intensities associated with the measurement path and the reference path.

Embodiment 15 includes the subject matter of any of Embodiment 14, and further specifies that the logic is configured to select a measurement path based on a CT image of the specimen.

Embodiment 16 includes the subject matter of any of Embodiments 14-15, and further specifies that the logic is configured to select a reference path based on a CT image of the specimen.

Embodiment 17 includes the subject matter of any of Embodiments 14-16, and further includes a gantry operable to rotate the x-ray source and the x-ray detector about the specimen to and direct the polychromatic, collimated x-ray along a plurality of measurement paths.

Embodiment 18 includes the subject matter of any of Embodiments 14-17, and further specifies that the logic is operable to determine tissue load by combining scattering cross-section values within a predetermined range of momentum transfer parameter values.

Embodiment 19 includes the subject matter of any of Embodiments 14-18, and further specifies that the predetermined range of momentum transfer parameter values corresponds to momentum transfer parameter values between 3.5 and 8.5 nm⁻¹.

Embodiment 20 includes the subject matter of any of Embodiments 14-19, and further specifies that the logic is operable to reconstruct a tissue load image and display the tissue load image superimposed with the CT image.

Embodiment 21 includes the subject matter of any of Embodiments 14-20, and further specifies that the at least one of the x-ray source and the x-ray detector are operable to produce CT data for generation of the CT image.

Embodiment 22 includes the subject matter of any of Embodiments 14-21, and further specifies that the x-ray detector is situated to receive scattered portions at angles of less than 5 degrees.

1. A method, comprising:
irradiating a region of interest (ROI) with a collimated, polychromatic x-ray beam;
in response to the irradiating, obtaining energy- and angle-resolved scattering intensities associated with a reference path and a measurement path;
processing the energy- and angle-resolved scattering intensities to produce scattering cross section as a function of a momentum transfer parameter; and
combining scattering cross-section within a predetermined range to determine amyloid burden.

2. The method of claim 1, wherein the predetermined range of momentum transfer parameter corresponds to momentum transfer parameter values between 3/nm and 9/nm, wherein q=4πE sin θ/hc, wherein E is x-ray energy, θ is a scattering angle, and hc is product of Planck's constant h and a vacuum speed of light c.

3. The method of claim 2, wherein the momentum transfer parameter is q.

4. The method of claim 1, further comprising selecting at least one of the reference path and the measurement path based on a CT image of the ROI.

5. The method of claim 1, further comprising selecting the reference path to avoid portions of the ROI associated with an amyloid load.

6. The method of claim 1, further comprising irradiating the region of interest (ROI) with the collimated, polychromatic x-ray beam along a plurality of measurement paths and for each measurement path:
processing associated energy- and angle-resolved scattering intensities to produce scattering cross section as a function of the momentum transfer parameter; and
combining each of the scattering cross-sections within the predetermined range to determine amyloid burden along each of the measurement paths.

7. The method of claim 6, further comprising irradiating the region of interest (ROI) with the collimated, polychromatic x-ray beam along reference paths associated with respective measurement paths for each measurement path, wherein the associated energy- and angle-resolved scattering intensities for the measurement paths and the respective reference paths are processed to produce scattering cross sections as a function of the momentum transfer parameter along each of the measurement paths.

8. The method of claim 1, wherein the ROI includes in vivo human brain and the amyloid burden is associated with the in vivo human brain.

9. The method of claim 6, wherein the irradiating the region of interest (ROI) with the collimated, polychromatic x-ray beam along a plurality of measurement paths comprises selecting a rotation of the collimated, polychromatic x-ray source and an oppositely situated x-ray detector about the ROI for each measurement path and obtaining energy- and angle-resolved scattering intensities associated with a reference path and a measurement path.

10. The method of claim 1, wherein the energy- and angle-resolved scattering intensities associated with a reference path and a measurement path are obtained with an x-ray detector array.

11. The method of claim 10, wherein the x-ray detector array comprises a plurality of individual x-ray detectors.

12. The method of claim 4, wherein the CT image is obtained with an uncollimated x-ray beam from an x-ray source, and further comprising situating at least one aperture with respect to the x-ray source to produce the collimated, polychromatic x-ray beam.

13. The method of claim 1, wherein polychromatic x-ray beam has x-ray photon energies between 30 and 80 keV.

14. An apparatus, comprising:
an x-ray source operable to produce a collimated, polychromatic x-ray beam and direct the collimated, polychromatic x-ray beam to a specimen;
an x-ray detector situated to receive scattered portions of the collimated, polychromatic x-ray beam from the specimen and produce signals corresponding to energy- and angle-resolved scattering intensities associated with a measurement path and a reference path through the specimen; and
logic configured to process the signals and produce an indication of a tissue load from a scattering cross-section dependence on a momentum transfer parameter, wherein the scattering cross section is based on the signals corresponding to the energy- and angle-resolved scattering intensities associated with the measurement path and the reference path.

15. The apparatus of claim 14, wherein the logic is configured to select a measurement path based on a CT image of the specimen.

16. The apparatus of claim 14, wherein the logic is configured to select a reference path based on a CT image of the specimen.

17. The apparatus of claim 14, further comprising a gantry operable to rotate the x-ray source and the x-ray detector about the specimen to and direct the polychromatic, collimated x-ray along a plurality of measurement paths.

18. The apparatus of claim 17, wherein the logic is operable to determine tissue load by combining scattering cross-section values within a predetermined range of momentum transfer parameter values.

19. The apparatus of claim 18, wherein the predetermined range of momentum transfer parameter values corresponds to momentum transfer parameter values between 3.5 and 8.5 nm⁻¹ or 7.3 and 9.7 nm⁻¹.

20. The apparatus of claim 17, wherein the logic is operable to reconstruct a tissue load image and display the tissue load image superimposed with a CT image.

21. The apparatus of claim 17, wherein at least one of the x-ray source and the x-ray detector are operable to produce CT data for generation of a CT image.

22. The apparatus of claim 17, wherein the x-ray detector is situated to receive scattered portions at angles of less than 5 degrees.
